(19) United States
(12) Patent Application Publication
(10) Pub. No.: US 2007/0021471 A1
(43) Pub. Date: Jan. 25, 2007

(54) Crystalline N-(2-((4-Hydroxyphenyl)amino)pyridin-3-yl)-4-methoxybenzenesulfonamide ethanolates

(76) Inventors: Jorge Gandarilla, North Riverside, IL (US); Karthryn L. Schardt, Grayslake, IL (US); John E. Quick, Waukegan, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/332,274

(22) Filed: Jan. 13, 2006

Related U.S. Application Data
(60) Provisional application No. 60/643,569, filed on Jan. 13, 2005.

(51) Int. Cl. A61K 31/44; C07D 213/72

(52) U.S. Cl. 514/352; 546/312

(57) ABSTRACT

Ethanolates of N-(2-((4-Hydroxyphenyl)amino)pyridin-3-yl)-4-methoxybenzenesulfonamide, ways to make them, compositions containing them or made with them, and methods of treating diseases using them or drugs made from them. are disclosed.

This application claims priority to co-pending U.S. Provisional Application Ser. No. 60/643,569, filed Jan. 13, 2005.

### FIELD OF THE INVENTION

This invention pertains to ethanolates of N-(2-((4-Hydroxyphenyl)amino)pyridin-3-yl)-4-methoxybenzenesulfonamide, ways to make them, compositions containing them or made with them, and methods of treating diseases using them or drugs made from them.

### BACKGROUND OF THE INVENTION

Physicial properties of drugs effects their manufacture and their utility. There is therefore an existing need in the chemical and therapeutic arts for identification of solvates of drugs and ways of reproducibly making them.

### SUMMARY OF THE INVENTION

One embodiment of this invention, therefore, pertains to crystalline N-(2-((4-hydroxyphenyl)amino)pyridin-3-yl)-4-methoxybenzenesulfonamide ethanolate.

Still another embodiment pertains to N-(2-((4-Hydroxyphenyl)amino)pyridin-3-yl)-4-methoxybenzenesulfonamide Ethanolate Crystalline Form 1 characterized, when measured at about 25° C. with Cu—Kα radiation, by a powder diffraction pattern with at least three peaks having respective 2θ values of about 8.4°, 9.3°, 12.8°, 13.1°, 16.1°, 17.0°, 17.9°, 18.7°, 20.0°, 20.1°, 21.2°, 24.4°, 25.5°, 25.8° or 26.0°.

Another embodiment pertains to N-(2-((4-Hydroxyphenyl)amino)pyridin-3-yl)-4-methoxybenzenesulfonamide Ethanolate Crystalline Form 1 characterized in the monoclinic crystal system, when measured at about 25° C. with Mo—Kα radiation, by respective lattice parameters a, b and c of 10.589 Å±0.0001 Å, 11.252 Å±0.0001 Å and 17.802 Å±0.0002 Å and β of 94.436°.

Still another embodiment pertains to crystalline N-(2-((4-Hydroxyphenyl)amino)pyridin-3-yl)-4-methoxybenzenesulfonamide ethanolate having substantial crystalline purity.

Still another embodiment pertains to N-(2-((4-Hydroxyphenyl)amino)pyridin-3-yl)-4-methoxybenzenesulfonamide Ethanolate Crystalline Form 1 having substantial crystalline purity and characterized, when measured at about 25° C. with Cu—Kα radiation, by a powder diffraction pattern with at least three peaks having respective 2θ values of about 8.4°, 9.3°, 12.8°, 13.1°, 16.1°, 17.0°, 17.9°, 18.7°, 20.0°, 20.1°, 21.2°, 24.4°, 25.5°, 25.8° or 26.0°.

Still another embodiment pertains to N-(2-((4-Hydroxyphenyl)amino)pyridin-3-yl)-4-methoxybenzenesulfonamide Ethanolate Crystalline Form 1 having substantial crystalline purity and characterized in the monoclinic crystal system, when measured at about 25° C. with Mo—Kα radiation, by respective lattice parameters a, b and c of 10.589 Å±0.0001 Å, 11.252 Å±0.0001 Å and 17.802 Å±0.0002 Å and β of 94.436°.

Still another embodiment pertains to crystalline N-(2-((4-hydroxyphenyl)amino)pyridin-3-yl)-4-methoxybenzenesulfonamide ethanolate having substantial crystalline purity and substantial chemical purity.

Still another embodiment pertains to N-(2-((4-Hydroxyphenyl)amino)pyridin-3-yl)-4-methoxybenzenesulfonamide Ethanolate Crystalline Form 1 having substantial crystalline purity and substantial chemical purity and characterized, when measured at about 25° C. with Cu—Kα radiation, by a powder diffraction pattern with at least three peaks having respective 2θ values of about 8.4°, 9.3°, 12.8°, 13.1°, 16.1°, 17.0°, 17.9°, 18.7°, 20.0°, 20.1°, 21.2°, 24.4°, 25.5°, 25.8° or 26.0°.

Still another embodiment pertains to N-(2-((4-Hydroxyphenyl)amino)pyridin-3-yl)-4-methoxybenzenesulfonamide Ethanolate Crystalline Form 1 having substantial crystalline purity and substantial chemical purity and characterized in the monoclinic crystal system, when measured at about 25° C. with Mo—Kα radiation, by respective lattice parameters a, b and c of 10.589 Å±0.0001 Å, 11.252 Å±0.0001 Å and 17.802 Å±0.0002 Å and β of 94.436°.

Still another embodiment pertains to compositions made with or comprising an excipient and a therapeutically acceptable amount of a crystalline N-(2-((4-hydroxyphenyl)amino)pyridin-3-yl)-4-methoxybenzenesulfonamide.

Still another embodiment pertains to methods for treating cancer in a mammal comprising administering thereto a therapeutically effective amount of a crystalline N-(2-((4-Hydroxyphenyl)amino)pyridin-3-yl)-4-methoxybenzenesulfonamide.

Still another embodiment pertains to processes for making N-(2-((4-Hydroxyphenyl)amino)pyridin-3-yl)-4-methoxybenzenesulfonamide Ethanolate Crystalline Form 1, said processes comprising:

providing a mixture consisting essentially of N-(2-((4-hydroxyphenyl)amino)pyridin-3-yl)-4-methoxybenzenesulfonamide and ethanol, with or without a solvent other than ethanol;

causing N-(2-((4-Hydroxyphenyl)amino)pyridin-3-yl)-4-methoxybenzenesulfonamide Ethanolate Crystalline Form 1 to exist in said mixture; and

isolating said crystalline N-(2-((4-Hydroxyphenyl)amino)pyridin-3-yl)-4-methoxybenzenesulfonamide Ethanolate Crystalline Form 1.

Still another em N-(2-((4-Hydroxyphenyl)amino)pyridin-3-yl)-4-methoxybenzenesulfonamide Ethanolate Crystalline Form 1, said processes comprising:

providing a mixture comprising N-(2-((4-hydroxyphenyl)amino)pyridin-3-yl)-4-methoxybenzenesulfonamide and ethanol, with or without a solvent other than ethanol;

causing crystalline N-(2-((4-hydroxyphenyl)amino)pyridin-3-yl)-4-methoxybenzenesulfonamide Ethanolate Crystalline Form 1 to exist in said mixture; and

isolating said N-(2-((4-Hydroxyphenyl)amino)pyridin-3-yl)-4-methoxybenzenesulfonamide Ethanolate Crystalline Form 1.

### DETAILED DESCRIPTION OF THE INVENTION

This invention pertains to N-(2-((4-hydroxyphenyl)amino)pyridin-3-yl)-4-methoxybenzenesulfonamide ethanolates, a particular example of which is crystalline N-(2-((4-hydroxyphenyl)amino)pyridin-3-yl)-4-methoxybenzenesulfonamide ethanolate Crystalline Form 1, a particular example of which is N-(2-((4-Hydroxyphenyl)amino)pyridin-3-yl)-4-methoxybenzenesulfonamide Ethanolate Crystalline Form 1 which may be characterized in the monoclinic crystal system, when measured at about 25° C. with Mo—Kα radiation, by respective lattice parameters a, b and c of about 10.589 Å±0.0001 Å, 11.252 Å±0.0001 Å and 17.802 Å±0.0002 Å and β of 94.436°, by a powder diffraction pattern, when measured at about 25° C. with Cu—Kα radiation, at least three peaks having respective 2θ values of about 8.4°, 9.3°, 12.8°, 13.1°, 16.1°, 17.0°, 17.9°, 18.7°, 20.0°, 20.1°, 21.2°, 24.4°, 25.5°, 25.8° or 26.0°, or by a combination thereof.

The term “crystalline,” as used herein, means having a regularly repeating arrangement of molecules or external face planes.

The term “substantial crystalline purity,” as used herein, means at least about 95% crystalline purity, preferably about 97% crystalline purity, more preferably about 99% crystalline purity, and most preferably about 100% crystalline purity.

The term “crystalline purity,” as used herein, means percentage of crystalline N-(2-((4-hydroxyphenyl)amino)pyridin-3-yl)-4-methoxybenzenesulfonamide ethanolate in a sample which may contain one or more than one other crystalline forms of N-(2-((4-hydroxyphenyl)amino)pyridin-3-yl)-4-methoxybenzenesulfonamide ethanolate.

The term “substantial chemical purity,” as used herein, means about 95% chemical purity, preferably about 97% chemical purity, more preferably about 98% chemical purity, and most preferably about 100% chemical purity.

The term “chemical purity,” as used herein, means percentage of N-(2-((4-hydroxyphenyl)amino)pyridin-3-yl)-4-methoxybenzenesulfonamide ethanolate in a sample. A sample of N-(2-((4-hydroxyphenyl)amino)pyridin-3-yl)-4-methoxybenzenesulfonamide ethanolate, may contain, for example, acetic acid, ethanol, ethyl acetate, isopropyl acetate, isopropyl ether, methanol, r-propanol, pyridine, pyridine hydrochloride, water, 4-aminophenol, 3,4-bis(4-hydroxyanilino)6-((4-hydroxyphenyl)imino)-2,4-cyclohexadien-1-one of varying geometric purity, 2-chloro-3-nitropyridine or a regioisomer thereof, 2,6-di-tert-butylphenol, 4-((3-nitro-2-pyridinyl)oxy)aniline, para-methoxybenzenesulfonyl chloride, 4-((3-(((4-methoxyphenyl)sulfonyl)amino)pyridin-2-yl)amino)phenyl 4-methoxybenzenesulfonate or a mixture thereof.

Unless stated otherwise, percentages herein are weight/weight (w/w) percentages.

The term “mixture,” as used herein, means a combination of two or more than two substances. For mixtures comprising or consisting essentially of an N-(2-((4-hydroxyphenyl)amino)pyridin-3-yl)-4-methoxybenzenesulfonamide and ethanol, with or without a solvent other than ethanol, the an N-(2-((4-hydroxyphenyl)amino)pyridin-3-yl)-4-methoxybenzenesulfonamide may be completely soluble or partially soluble in the solvent.

It is meant to be understood that solvent molecules from solvated N-(2-((4-hydroxyphenyl)amino)pyridin-3-yl)-4-methoxybenzenesulfonamide may be used as solvent for preparation of a crystalline N-(2-((4-hydroxyphenyl)amino)pyridin-3-yl)-4-methoxybenzenesulfonamide ethanolate.

The term “solvate,” as used herein, means including a solvent such as acetic acid, acetone, acetonitrile, benzene, chloroform, carbon tetrachloride, dichloromethane, dimethylsulfoxide, 1,4-dioxane, ethanol, ethyl acetate, butanol, tert-butanol, N,N-dimethylacetamide, N,N-dimethylformamide, formamide, formic acid, heptane, hexane, isopropanol, methanol, 1-methyl-2-pyrrolidinone, mesitylene, nitromethane, polyethylene glycol, propanol, 2-propanone, pyridine, tetrahydrofuran, toluene, water, xylene, or a mixture thereof.

Causing a crystalline N-(2-((4-hydroxyphenyl)amino)pyridin-3-yl)-4-methoxybenzenesulfonamide ethanolate to exist in a mixture comprising N-(2-((4-hydroxyphenyl)amino)pyridin-3-yl)-4-methoxybenzenesulfonamide and ethanol, with or without a solvent other than ethanol, wherein the N-(2-((4-hydroxyphenyl)amino)pyridin-3-yl)-4-methoxybenzenesulfonamide is completely soluble in the solvent, is known as nucleation.

Nucleation may be made to occur by means such as solvent removal, temperature change, solvent-miscible anti-solvent addition, solvent-immiscible anti-solvent addition, chafing or scratching the interior of the container, preferably a glass container, in which nucleation is meant to occur with an implement such as a glass rod or a glass bead or beads, or a combination of the foregoing.

The term “solvent,” as used herein, means a substance, preferably a liquid or a miscible, partially miscible or essentially immiscible mixture of two or more than two liquids, which is capable of completely dissolving, partially dissolving, dispersing or partially dispersing another substance, preferably a solid or a mixture of solids.

The term “miscible,” as used herein, means capable of combining without separation of phases.

The term “anti-solvent,” as used herein, means a solvent in which N-(2-((4-hydroxyphenyl)amino)pyridin-3-yl)-4-methoxybenzenesulfonamide ethanolate is essentially insoluble at a particular temperature or concentration.

It is meant to be understood that, because many solvents and anti-solvents contain impurities, the level of impurities in solvents and anti-solvents for the practice of this invention, if present, are at a low enough percentage that they do not interfere with the intended use of the solvent in which they are present.

The term “isolating” or “isolation,” as used herein, means separating an N-(2-((4-hydroxyphenyl)amino)pyridin-3-yl)-4-methoxybenzenesulfonamide ethanolate and impurity, wherein the impurity may be solvent, anti-solvent, a solid or a mixture thereof. Isolation is typically accomplished by means such as centrifugation, filtration with or without vacuum, filtration under positive pressure, distillation, evaporation or a combination thereof.

An exemplary N-(2-((4-hydroxyphenyl)amino)pyridin-3-yl)-4-methoxybenzenesulfonamide ethanolate may be made by the procedures described hereinbelow.

N-(2-((4-hydroxyphenyl)amino)pyridin-3-yl)-4-methoxybenzenesulfonamide may also be written as R¹SO₂NHR², wherein R is 4-methoxyphenyl and R² is 2-((4-hydroxyphenyl)aminopyridin-3-yl.

### EXAMPLE 1

A mixture of 2-chloro-3-nitropyridine (2C3NP, 138.1 Kg), 4-aminophenol (2.5-3 equivalents) and N,N-dimethylformamide (DMF, 4.8 mL/g 2C3NP) was stirred until homogeneous, heated at 50° C. during which an exotherm raised the solution temperature to 70° C., warmed to 80-85° C., stirred until no 2-chloro-3-nitropyridine remained, cooled to 30° C., treated with water (10.6 mL/g 2C3NP) to precipitate product, then with acetic acid (1.2 mL/g 2C3NP), then with ethyl acetate (0.5 mL/g 2C3NP), cooled to 5° C., stirred for 2 hours and filtered. The filtrant was washed sequentially with distilled water (1.6 mL/g 2C3NP), cold ethanol (1.2 mL/g 2C3NP) and cold isopropyl ether (1.2 mL/g 2C3NP), and dried under vacuum.

In a preferred embodiment of this process, 4-aminophenol (1 equivalent) was used with 4-methylmorpholine (1.5 equivalents) in either methanol or DMF, and precipitation was accomplished with 10% aqueous acetic acid.

### EXAMPLE 2

A mixture of EXAMPLE 1 (41.05 Kg) and ammonium formate (5 equivalents), with or without 2,6-di-tert-butylphenol antioxidant, was treated with a mixture of 50% wet 5% palladium hydroxide on carbon (7% by weight per weight of EXAMPLE 1), in DMF (6 mL/g catalyst) then DMF (total DMF volume: 5 mL/g EXAMPLE 1) first with moderate agitation to control an exotherm (typically peaking at 85° C.) then with increased agitation for 1 hour (incomplete reactions were treated with additional catalyst/DMF mixture), cooled to 10° C., and filtered. The filtrant was washed with DMF (0.4 mL/g EXAMPLE 1), and the filtrate was added to water (29.4 mL/g EXAMPLE 1) at 10° C. to precipitate a solid which was filtered, washed with water (7.5 mL/g EXAMPLE 1), partially dried under a nitrogen stream, and further dried under vacuum at 50° C. to about 0.5% moisture.

### EXAMPLE 3

A mixture of EXAMPLE 2 in pyridine (9 mL/g) at 0° C. was treated with a mixture of para-methoxybenzenesulfonyl chloride (1.05 equivalents) in THF (1.4 mL/g) at 0° C. at a rate which kept the reaction temperature below 5° C., warmed to 25° C., stirred for 15 minutes, and concentrated. The concentrate was treated with n-propanol to provide a composition having 9% pyridine in the solvent mixture and to precipitate a solid. The mixture was cooled to 0° C. and filtered. The filtrant and washed with ethyl acetate (5-7 mL/g starting material) and dried at 45° C.

### EXAMPLE 4

A mixture of EXAMPLE 3 and saturated aqueous sodium bicarbonate (2 equivalents) was extracted with ethyl acetate (6 mL/g EXAMPLE 3). The extract was washed with brine (4 mL/g EXAMPLE 3), treated with n-propanol (2 mL/g EXAMPLE 4), and concentrated until the ethyl acetate was present in less than 1%. The concentrate was adjusted to 70:30 n-propanol:water (150-180 mg EXAMPLE 4/g solution), and the hot solution was filtered through a 0.2 micrometer filter. The filtrate was cooled as quickly as possible to 0° C., adjusted to a solvent composition of 1:1 n-propanol:water, allowed to stand until the amount of R¹SO₂NHR² stabilized, and filtered. The filtrant was washed with 40:60 n-propanol:water (1.8 Kg/Kg R¹SO₂NHR²) and dried at 45° C. under vacuum.

An N-(2-((4-hydroxyphenyl)amino)pyridin-3-yl)-4-methoxybenzenesulfonamide ethanolate may be made by recrystallizing EXAMPLE 4 from ethanol, with or without a solvent other than ethanol.

Exemplary N-(2-((4-Hydroxyphenyl)amino)pyridin-3-yl)-4-methoxybenzenesulfonamide Ethanolate Crystalline Form 1, for powder diffraction analysis, was applied as a thin layer, with no prior grinding, to the analysis well of a Scintag XDS 2000 Diffractometer having the following parameters: x-ray source: Cu—Kα; range: 2.00°-40.00° 2θ; scan rate: 1.00 degree per minute; step size: 0.02°; temperature: about 25° C.; wavelength: 1.54178 Å.

Representative characteristic peak positions in the powder diffraction pattern of N-(2-((4-Hydroxyphenyl)amino)pyridin-3-yl)-4-methoxybenzenesulfonamide Ethanolate Crystalline Form 1, expressed as degrees relative to 2θ, are, when measured at about 25° C. with Cu—Kα radiation, about 8.4°(1,0,0), 9.3° (0,1,1), 12.8°(0,1,2), 13.1°(1,1,1), 16.1°(1,1,2), 17.0°(0,1,3), 17.9°(1,2,0), 18.7°(2,1,0), 20.0°(1,1,3), 20.1°(1,0,-4), 21.2°(1,2,2), 24.4°(1,1,4), 25.5°(1,3,-1), 25.7°(0,2,4), 25.8°(0,3,2) or 26.0°(1,3,1). Each peak position is shown with its accompanying Miller index (hkl) values.

The term “about” preceding a series of peak positions is meant to include all of the peak positions of the group which the term precedes.

It is meant to be understood that peak heights may vary and will be dependent on variables such as the temperature, size of crystal size or morphology, sample preparation, or sample height in the analysis well of the diffractometer.

It is also meant to be understood that peak positions may vary when measured with different radiation sources. For example, Cu—Kα₁, Mo—Kα, Co—Kα and Fe—Kα radiation, having wavelengths of 1.54060 Å, 0.7107 Å, 1.7902 Å and 1.9373 Å, respectively, may provide peak positions which differ from those measured with Cu—Kα radiation.

While digital outputs from powder x-ray diffractometers may be set to express peak positions to the one-hundredth and one-thousandth of a degree past the decimal, diffractometers are incapable of accurate experimental determination beyond one-tenth of a degree. Accordingly, peak positions reported herein are rounded to one-tenth of a degree past the decimal.

Peak positions may also be expressed with a variability which accounts for differences between powder x-ray diffractometers, and variability between Cu—Kα radiation sources, variability from sample to sample on the same diffractometer, and differences in sample heights in the analysis well. This variability is preferably expressed as about ±0.2°, about ±0.1°, or a combination thereof.

N-(2-((4-Hydroxyphenyl)amino)pyridin-3-yl)-4-methoxybenzenesulfonamide Ethanolate Crystalline Form 1, when measured in the monoclinic crystal system at about 25° C. with Cu—Kα radiation, is also characterized by when measured at about 25° C. with Mo—Kα radiation, by respective lattice parameters a, b and c of about 10.589 Å±0.0001 Å, 11.252 Å±0.000 Å and 17.802 Å±0.0002 Å and β of 94.436°. Because this monoclinic crystal system has a regularly repeating arrangement of molecules, N-(2-((4-Hydroxyphenyl)amino)pyridin-3-yl)-4-methoxybenzenesulfonamide Ethanolate Crystalline Form 1 may also be characterized by its space group (P2₁/c) in addition to at least three peak positions in its powder diffraction, such as, for example, at least three peaks having respective 2θ values of about 8.4°, 9.3°, 12.8°, 13.1°, 16.1°, 17.0°, 17.9°, 18.7°, 20.0°, 20.1°, 21.2°, 24.4°, 25.5°, 25.8° or 26.0° when measured at about 25° C. with Cu—Kα radiation.

The utility of N-(2-((4-Hydroxyphenyl)amino)pyridin-3-yl)-4-methoxybenzenesulfonamide Ethanolate Crystalline Form 1 is demonstrated in commonly-owned U.S. application Ser. No. 10/857,235, May 28, 2004 and U.S. Application Ser. No. 60/575,577, May 28, 2004.

N-(2-((4-Hydroxyphenyl)amino)pyridin-3-yl)-4-methoxybenzenesulfonamide binds to the colchicine site of tubulin β-subunits and inhibits the polymerization of tubulin. Accordingly, the compound is useful as a drug for treating diseases in a mammal which are caused or exacerbated by polymerization of tubulin. Such diseases include, but are not limited to, cancer and gouty arthritis, wherein cancer includes, but is not limited to, bone marrow dyscrasias, breast (ductal and lobular) cancer, cervical cancer, colon cancer, leukemia, lung (small cell and non-small cell) cancer, lymphoma, melonoma, mouth and tongue cancer, neuroblastoma (including pediatric neuroblastoma), pancreatic cancer, prostate cancer, rectal cancer, renal cancer, sarcoma, stomach cancer, uterine cancer, and cancers resulting from the metastasis of disease from these areas.

The term “mammal,” as used herein, means a particular class of vertebrate, preferably a human.

Compositions made with or comprising an N-(2-((4-hydroxyphenyl)amino)pyridin-3-yl)-4-methoxybenzenesulfonamide ethanolate may be administered, for example, bucally, ophthalmically, orally, osmotically, parenterally (intramuscularly, intraperintoneally, intrasternally, intravenously, subcutaneously), rectally, topically, transdermally, or vaginally. Ophthalmically administered dosage forms may be administered as, for example, elixirs, emulsions, microemulsions, oinments, solutions, suspensions, or syrups. Orally administered solid dosage forms may be administered as, for example, capsules, dragees, emulsions, granules, pills, powders, solutions, suspensions, tablets, microemulsions, elixirs, syrups, or powders for reconstitution. Osmotically and topically administered dosage forms may be administered as, for example, creams, gels, inhalants, lotions, ointments, pastes, or powders. Parenterally administered dosage forms may be administered, as, for example, aqueous or oleaginous solutions or suspensions. Rectally and vaginally dosage forms may be administered as, for example, creams, gels, lotions, ointments or pastes.

The therapeutically acceptable amount of an N-(2-((4-hydroxyphenyl)amino)pyridin-3-yl)-4-methoxybenzenesulfonamide ethanolate depends on recipient of treatment, the disease and severity thereof, the composition containing it, time of administration, route of administration, duration of treatment, its potency, its rate of clearance and whether or not another drug is co-administered. The amount of an N-(2-((4-hydroxyphenyl)amino)pyridin-3-yl)-4-methoxybenzenesulfonamide ethanolate used to make a composition to be administered daily to a patient in a single dose or in divided doses is from about 0.03 to about 200 mg/kg body weight. Single dose compositions contain these amounts or a combination of submultiples thereof.

An N-(2-((4-hydroxyphenyl)amino)pyridin-3-yl)-4-methoxybenzenesulfonamide ethanolate may be administered with or without an excipient. Excipients include, but are not limited to, encapsulating materials and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof.

Excipients for preparation of compositions comprising or made with an N-(2-((4-hydroxyphenyl)amino)pyridin-3-yl)-4-methoxybenzenesulfonamide ethanolate to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof. Excipients for preparation of compositions comprising or made with an N-(2-((4-hydroxyphenyl)amino)pyridin-3-yl)-4-methoxybenzenesulfonamide ethanolate to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, and mixtures thereof. Excipients for preparation of compositions made with or comprising an N-(2-((4-hydroxyphenyl)amino)pyridin-3-yl)-4-methoxybenzenesulfonamide ethanolate to be administered osmotically include, for example, chlorofluorohydrocarbons, ethanol, water, and mixtures thereof. Excipients for preparation of compositions made with or comprising an N-(2-((4-hydroxyphenyl)amino)pyridin-3-yl)-4-methoxybenzenesulfonamide ethanolate to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for preparation of compositions made with or comprising an N-(2-((4-hydroxyphenyl)amino)pyridin-3-yl)-4-methoxybenzenesulfonamide ethanolate to be administered rectally or vaginally include, for example, cocoa butter, polyethylene glycol, wax, and mixtures thereof.

The foregoing is meant to be illustrative of the invention and not intended to limit it to the embodiments disclosed herein. Variations and changes obvious to one skilled in the art are intended to be within the scope and nature of the invention as defined in the claims.

What is claimed is:

1. Crystalline N-(2-((4-hydroxyphenyl)amino)pyridin-3-yl)-4-methoxybenzenesulfonamide ethanolate.

2. N-(2-((4-Hydroxyphenyl)amino)pyridin-3-yl)-4-methoxybenzenesulfonamide Ethanolate Crystalline Form 1 characterized, when measured at about 25° C. with Cu—Kα radiation, by a powder diffraction pattern with at least three peaks having respective 2θ values of about 8.4°, 9.3°, 12.8°, 13.1°, 16.1°, 17.0°, 17.9°, 18.7°, 20.0°, 20.1°, 21.2°, 24.4°, 25.5°, 25.8° or 26.0°.

3. A composition made with or comprising an excipient and a therapeutically acceptable amount of a crystalline N-(2-((4-hydroxyphenyl)amino)pyridin-3-yl)-4-methoxybenzenesulfonamide.

4. A method for treating cancer in a mammal comprising administering thereto a therapeutically effective amount of a crystalline N-(2-((4-hydroxyphenyl)amino)pyridin-3-yl)-4-methoxybenzenesulfonamide.

5. A processes for making N-(2-((4-Hydroxyphenyl)amino)pyridin-3-yl)-4-methoxybenzenesulfonamide Ethanolate Crystalline Form 1, said processes comprising:
providing a mixture consisting essentially of N-(2-((4-hydroxyphenyl)amino)pyridin-3-yl)-4-methoxybenzenesulfonamide and ethanol, with or without a solvent other than ethanol;
causing N-(2-((4-Hydroxyphenyl)amino)pyridin-3-yl)-4-methoxybenzenesulfonamide Ethanolate Crystalline Form 1 to exist in said mixture; and
isolating said crystalline N-(2-((4-Hydroxyphenyl)amino)pyridin-3-yl)-4-methoxybenzenesulfonamide Ethanolate Crystalline Form 1.
